(19) United States
(12) Patent Application Publication
(10) Pub. No.: US 2004/0082867 A1
(43) Pub. Date: Apr. 29, 2004

(54) Vascular graft with integrated sensor

(75) Inventors: Victor C. Esch, San Francisco, CA (US); Luiz B. Da Silva, Danville, CA (US); Paul J. Weber, Fort Lauderdale, FL (US)

Correspondence Address: John P. Wooldridge, 1334 Ridegestone Ct., Livermore, CA, 94551, US

(73) Assignee: Pearl Technology Holdings, LLC

(21) Appl. No.: 10/619,289

(22) Filed: Jul. 15, 2003

Related U.S. Application Data
(60) Provisional application No. 60/421,981, filed on Oct. 29, 2002.

(51) Int. Cl. A61B 5/02; A61F 2/06

(52) U.S. Cl. 600/488; 623/912; 128/903

(57) ABSTRACT

A noninvasive medical device and diagnostic system is disclosed that can be used to determine the degree of viability, flow and stenosis of a graft within the human body. This invention incorporates a sensing element in or near an AV graft. As stenosis occurs within or near the graft, the change in pressure within the graft can be detected by the sensors and provide the physician with information about the location of the stenosis. This information can than be used to provide the best course of treatment for the patient.

This application claims priority to Provisional Patent Application Serial No. 60/421,981, titled “ARTERIOVENOUS GRAFT WITH INTEGRATED SENSOR”, filed Oct. 29, 2002 and incorporated herein by reference.

### BACKGROUND OF THE INVENTION

1. Field of the Invention

The present invention relates to techniques for monitoring arteriovenous integrity, and more specifically, it relates to a noninvasive medical device and diagnostic system for determining the degree of viability, flow and stenosis of an arteriovenous graft within the human body.

2. Description of Related Art

Synthetic vascular grafts have been used in medicine for over 30 years. They are used to replaced diseased arteries and veins. A variety of different materials and configurations have been developed and used. The goal is to produce a synthetic graft that has mechanical properties very similar to normal human arteries or veins. In addition, the synthetic graft needs to be biocompatible and have compliance similar to native arteries. Successful synthetic grafts have been developed out of ePTFE (See, e.g., U.S. Pat. No. 6,428,571 and U.S. Pat. No. 5,800,512) and polyurethane (See, e.g., U.S. Pat. No. 4,731,073). Ideally these grafts remain unobstructed and patent indefinitely without any clot or generation of emboli. However, in many cases stenosis or thrombosis occurs within and outside the graft leading to a significant reduction in blood flow and serious risks to the patient. Diagnostic techniques such as ultrasound imaging, or x-ray angiography are now used to detect stenosis. These techniques are time consuming and expensive to perform. A vascular graft with integrated sensors has been described by Weissman et al. (WO 02/00118 A2), however, their design requires a communicating element to communicate sensor measurements to an external remote detector. The need for a communicating element increases the complexity and cost of the graft. The implantable device described by Spillman et al. (U.S. Pat. No. 6,206,835 B1) has a single passive sensing circuit is that uses a single sensor that reduces the sensitivity to stenosis.

A need exists for a synthetic graft with integrated passive sensors that can quickly identify early stage stenosis allowing physicians to effectively reduce vascular access complications. The present invention fulfills this need, and further provides related advantages.

### SUMMARY OF THE INVENTION

An object of the present invention is to provide a synthetic vascular graft with integrated sensors that can be remotely interrogated to determine the degree of occlusion within or near the synthetic graft.

Another object of the present invention is to provide a synthetic vascular graft with integrated sensors that can be remotely interrogated to determine the location of an occlusion within or near the synthetic graft.

These and other objects will be apparent to those skilled in the art based on the disclosure herein.

It is commonly held that the surveillance of the status of access grafts and fistula is critical to the proper care of a dialysis patient. It is very important to diagnose failing vascular access prior to occlusion. Occlusion can occur due to cellular growth or thrombosis. In both cases, the result can lead to total occlusion and subsequent graft failure that may require emergency care and surgery. Sullivan et al. (Sullivan K L, Besarab A, Bonn J, Shapiro M J, Gardiner G A, Moritz M J, Hemodynamics of Failing Dialysis Grafts, Radiology 1993; 186:867, incorporated herein by reference) have shown that intragraft blood pressure measurements can be used to detect occlusions. In particular, simultaneous measurements of the blood pressure at the arterial and venous end of the graft can be used to quantify the degree and location of stenosis. The present invention uses this concept to develop an implantable synthetic graft that can be externally interrogated.

In one embodiment of the present invention, two passive pressure sensor elements are integrated one near each end of a single layer synthetic graft. The synthetic graft is made of a single layer of a biocompatible polymer (e.g., expanded polytetrafluorethylene (ePTFE), polyurethane). The passive pressure sensors are comprised of a capacitive element, C, and an inductive element, L, that forms an LC circuit. The impedance of this circuit becomes totally resistive at the characteristic resonance frequency, ω=1/\sqrt{LC}. The sensors are designed such that the resonance frequency changes as the blood pressure within the graft changes. The range of the resonance frequency of the proximal and distal sensors can be different to easily separate the signals during measurement. An external pickup coil detects the resonance frequency and therefore measures the pressure. The sensor is designed to have a response time of less than 100 msec to allow the complete cardiac pressure pulse to be accurately measured. The resonance frequency of each sensor should be in the range of 1 MHz to 200 MHz to reduce sensor size and operate where tissue has low conductivity.

In an alternative embodiment of the present invention, two passive pressure sensors are integrated, one near each end of a multilayer synthetic graft. The synthetic graft is made of at least two layers of a biocompatible polymer (e.g., ePTFE, polyurethane). The synthetic graft layers can have different mechanical properties and porosity. U.S. Pat. No. 6,428,571 by Lentz et al., incorporated herein by reference, provides an example of a multilayer vascular graft that can be modified to include passive pressure sensors. In a multilayer synthetic graft, the sensors can be placed between layers to simplify manufacturing.

In one embodiment of the passive pressure sensor, the capacitive element is a cylindrical capacitor whose spacing between the plates is a function of the intragraft blood pressure. In another embodiment of the passive pressure sensor, the capacitive element is a split cylindrical capacitor. In order to increase the pressure sensitivity, the dielectric between the capacitors can be a trapped fluid (e.g., water, silicone).

In one embodiment of the passive sensor, the inductive element is a coil that wraps around the tubular graft. In another embodiment of the passive sensor, the inductive element is a coil placed on the top surface of the tubular graft with its axis perpendicular to the tubular graft axis.

In an alternative embodiment of the present invention, the passive pressure sensors are optical pressure sensors whose optical properties change as a function of intragraft blood pressure. One possible optical sensor is a Fabry-Perot filter element whose mirror spacing changes with pressure. In this embodiment, an external broadband light source illuminates the sensor area and the returned optical signal is measured with a spectrometer to detect the operating wavelength of the filter. Depending on the filter design, the returned signal can have a maximum or minimum at the filter operating wavelength. In order to reduce the effects of tissue absorption, the operating wavelength of the system should be in the range between 600 nm and 1200 nm. In this spectral range, the scattering properties of tissue are smooth functions, which make it possible to subtract the large background signal to detect the comparatively weak narrowband filter signal. In one embodiment, the operating wavelength of the distal and proximal sensors can be different to simplify analysis.

In a similar embodiment, the external light source is used to excite a fluorescent material, the fluorescent signal then interacts with the filter, allowing detection of pressure characteristics. This has the advantage of allowing an excitation source that does not contribute to background signal in the detection wavelength range. This dramatically increases signal to noise. Fluorescent material peak response, or filter response, can be selected to allow easy separate detection of multiple detection sites. Alternatively, the filter can be designed to act upon the excitation wavelength only, with essentially no effect upon the fluorescent wavelength, and thus the fluorescent signal is indicative of the amount of excitation passed by the filter, and thereby the local pressure signal.

The present invention also relates to a method to detect occlusions within or near a synthetic vascular graft. An external detector is placed over the pressure sensors that are integrated into the synthetic graft and the blood pressure measured during a minimum of one cardiac cycle. A microprocessor in the external detector analyzes the pressure pulse measurements from the proximal and distal sensors and produces an output indicative of the condition of the graft. This output can be used by physicians to identify the need for intervention to prevent total graft occlusion.

The passive pressure sensors measure a pressure that is related to the blood pressure within the graft. Laboratory calibration of the sensors can be used to correct for the effects of a polymer layer between the blood and the sensor element. When the graft is implanted and cellular growth into the graft changes the mechanical properties, small errors could be introduced in converting from sensor pressure to blood pressure. The use of a proximal and distal sensor allows for ratio measurements to be performed that can reduce the sensitivity to changes in graft properties.

In addition, to hemodialysis grafts, it may be appreciated that there are many applications where this technology may yield useful information, including CABG grafts, and peripheral vascular grafts.

### BRIEF DESCRIPTION OF THE DRAWINGS

The accompanying drawings, which are incorporated into and form part of this disclosure, illustrate embodiments of the invention and together with the description, serve to explain the principles of the invention.

FIG. 1 is an illustration of an embodiment of the present invention having a vascular graft with integrated pressure sensors.

FIG. 2 is cross sectional view of one embodiment of the sensor section of a single layer synthetic graft.

FIG. 3 is cross sectional view of an alternative embodiment of the sensor section of a single layer synthetic graft.

FIG. 4 is cross sectional view of an alternative embodiment of the sensor section of a single layer synthetic graft.

FIG. 5 is cross sectional view of an alternative embodiment of the sensor section that uses a fluid cell to increase the sensitivity.

FIG. 6 is cross sectional view of an alternative embodiment with an optical pressure sensor that uses a Fabry-Perot filter concept.

FIG. 7 is cross sectional view of an alternative optical pressure sensor that uses an absorbing fluid layer.

FIG. 8 is cross sectional view of one embodiment of the sensor section in a two layer synthetic graft.

FIG. 9 is cross sectional view of one embodiment of the sensor section in a three layer synthetic graft.

FIG. 10 shows the effective sensing and sensor circuit.

FIG. 11 shows the measured resonance frequency of a prototype sensor for a fixed intragraft pressure of 150 mm Hg.

FIG. 12 is an illustration showing the key components of the measurement system.

FIG. 13 shows the pressure pulse as measured with the present invention.

### DETAILED DESCRIPTION OF THE INVENTION

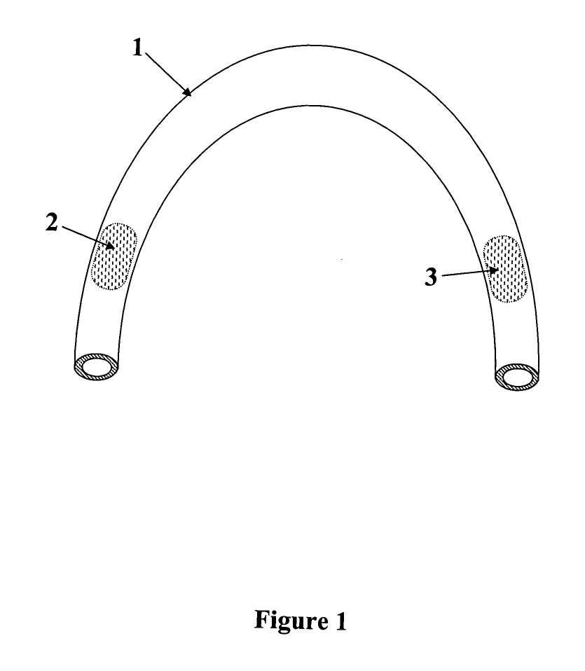

FIG. 1 is an illustration of one embodiment of the synthetic graft. One end of the graft 1 has a pressure sensor 2 integrated near the end of the graft, leaving enough material for suturing the graft to the vein or artery. The other end of the graft also contains an integrated pressure sensor 3 located near the end of the graft. In an alternative embodiment, one or both pressure sensors are located away from the end of the graft to allow the surgeon to cut the graft to the optimum length. The ends of the graft could also be flared or shaped to improve suturing. The graft can be made to any length but will typically be in the range of 5 cm to 50 cm. The sensors will each occupy less than 2 cm of length on the graft. When used as a hemodialysis graft the area between the sensors can be used to provide needle access to the patient's blood.

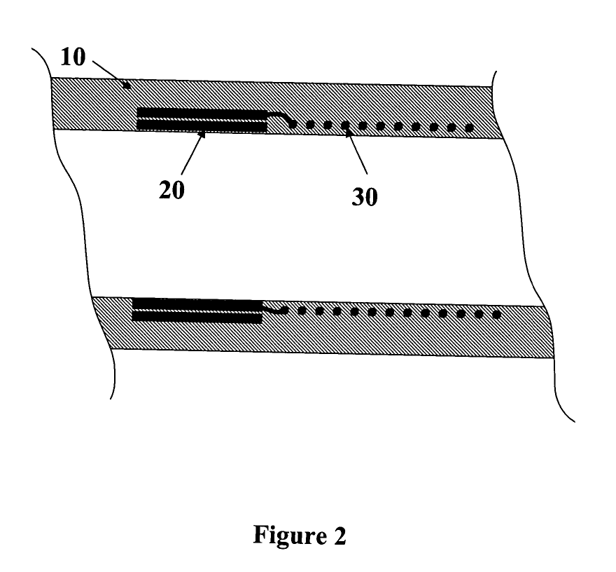

FIG. 2 shows a cross sectional view through the sensor area of one embodiment of the graft. The sensor elements are imbedded within a single layer of biocompatible material 10 (e.g., expanded-polytetraflouroethylene (ePTFE), polyurethane). The sensor consists of a capacitive pressure sensor 20 and an inductive coil 30 that forms a passive LC circuit. During the cardiac cycle, the capacitance of the pressure sensor chances causing a change in the resonance frequency of the LC circuit. In this embodiment, the pressure sensor 20 is a cylindrical capacitor with the plates separated by an air gap or compressible dielectric. In an alternative embodiment, the cylindrical capacitor is replaced by a split ring capacitor. The inductive coil 30 loops within the single layer of biocompatible material 10 of the graft and acts as the main coupling element to the external remote detector. In one embodiment, the sensor elements 20 and 30 are encapsulated with a non-conductive, impermeable biocompatible polymer to eliminate electrical short circuits of the elements.

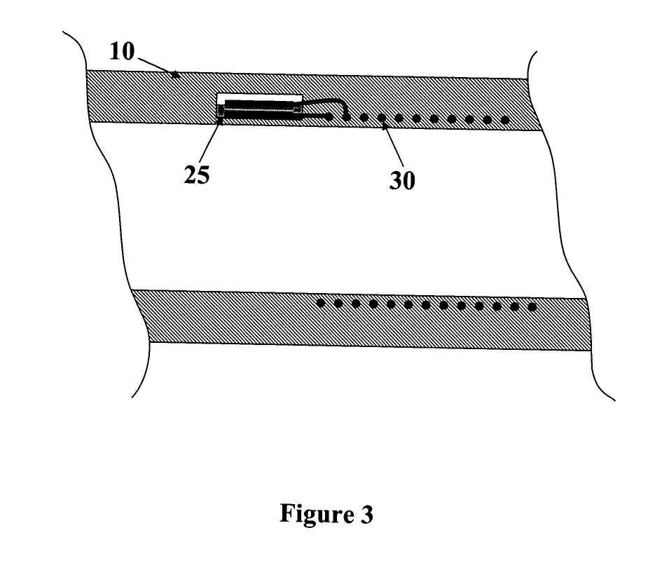

FIG. 3 shows a cross sectional view through the sensor area of an alternative embodiment of the graft. The sensor elements are imbedded within a single layer of biocompatible material 10 (e.g., expanded-polytetraflouroethylene (e-PTFE), polyurethane). The sensor consists of a capacitive pressure sensor 25 and an inductive coil 30 that forms a passive LC circuit. During the cardiac cycle, the capacitance of the pressure sensor chances causing a change in the resonance frequency of the LC circuit. In this embodiment, the pressure sensor 25 is a miniature parallel plate capacitive pressure sensor. This configuration has the possible drawback of being susceptible to local stenosis that could affect the measurement. The inductive coil 30 loops within the single layer of biocompatible material 10 of the graft and acts as the main coupling element to the external remote detector.

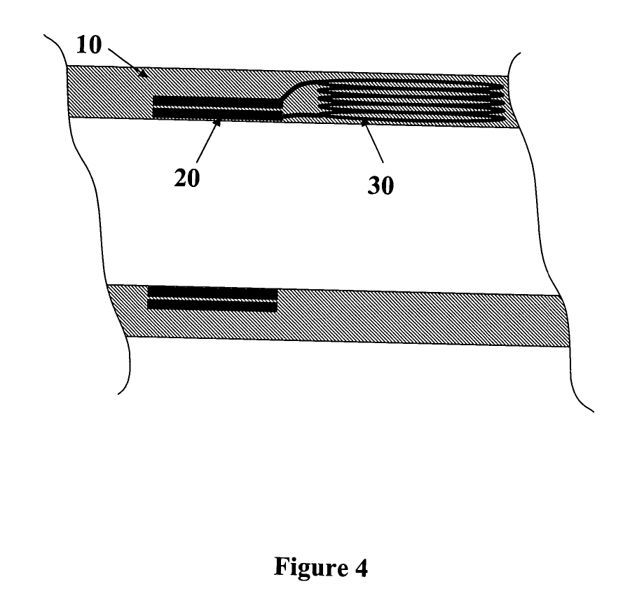

FIG. 4 shows a cross sectional view through the sensor area of an alternative embodiment of the graft. In this embodiment, the inductive element 30 is a coil whose central axis is approximately perpendicular to the central axis of the graft. This design can increase the mutual inductance to an external pickup coil.

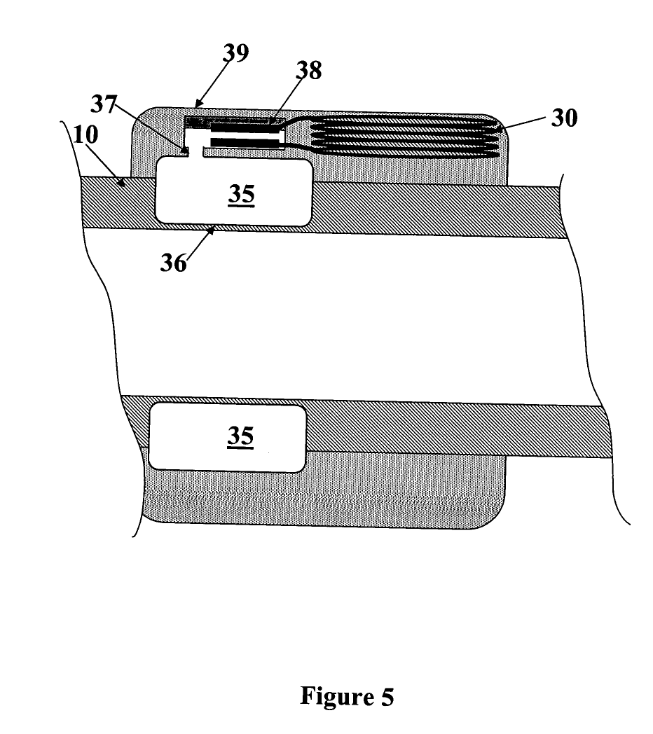

FIG. 5 is cross sectional view of an alternative embodiment of the sensor section that uses a fluid cell 35 to increase the sensitivity. Small changes in the volume of fluid cell 35 due to changes in the intragraft pressure that drives surface 36 can lead to significant volume changes in the capacitive sensor 38. Fluid moves into the capacitive sensor 38 through channel 37. The fluid can be any non-conductive biocompatible fluid such as water, silicone, biocompatible oils. The sensor elements are encapsulated within a semi-rigid biocompatible polymer ring 39. The polymer ring 39 can be bonded to the synthetic graft 10 chemically or during molding of the graft. The capacitive sensor 38 connects to inductive coil 30′ to form the resonant LC circuit.

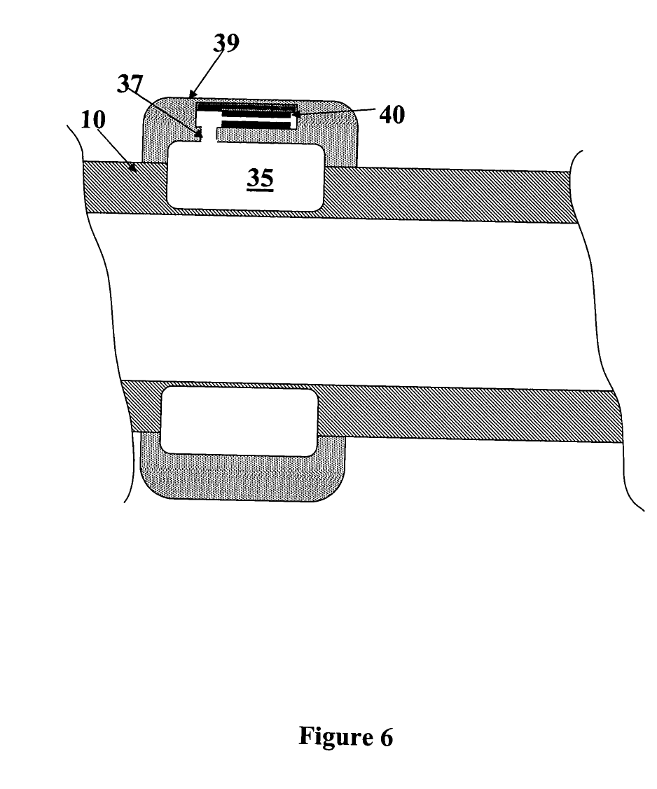

FIG. 6 is cross sectional view of an optical pressure sensor that uses a Fabry-Perot filter concept. As in the LC circuit of FIG. 5, this optical pressure sensor uses a fluid cell to change the spacing between the Fabry-Perot mirrors 40. An alternative embodiment would eliminate the fluid cell and place the optical sensor in a similar position to that of capacitor 25 illustrated in FIG. 3. Using an optical sensor eliminates the need for an inductive coil since light is used to probe the sensor. In order to reduce the effects of tissue absorption, the operating wavelength of the filter should be in the range between 600 nm and 1200 nm. In this spectral range the scattering properties of tissue are smooth functions that make it possible to subtract the large background signal to detect the comparatively weak narrowband filter signal. In one embodiment, the operating wavelength of the distal and proximal sensors can be different to simplify analysis. In this embodiment the external measurement device illuminates the sensor and detects the reflected signal.

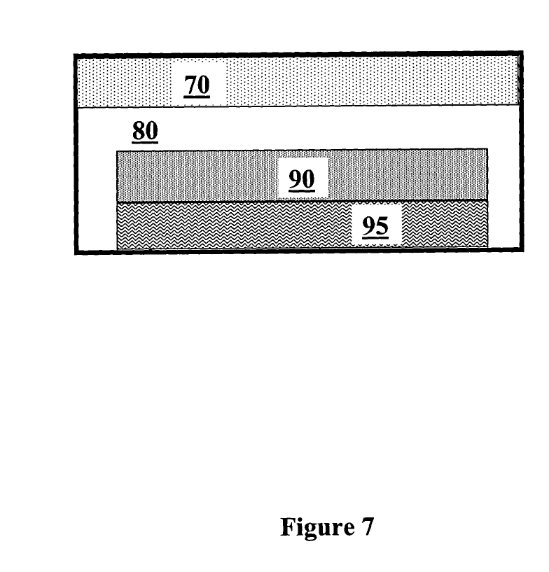

FIG. 7 shows a cross sectional view of an alternative optical pressure sensor that can be integrated into the graft as shown in FIG. 6. In this optical sensor, transparent polymer 70 and fluorescent polymer 90 are separated by fluid 80 that preferentially absorbs the excitation light or the fluorescence emission from the fluorescent polymer 90. As the separation between the transparent polymer 70 and the fluorescent polymer 90 changes, the fluorescence intensity will also change due to changes in absorption through the fluid 80. In order to account for other sources of changes in the fluorescence intensity (e.g., changes in separation between sensor and detector) the fluorescent polymer 90 could include a second fluorescent dye that emits light at a wavelength not absorbed by the fluid. Alternatively, the transparent polymer 70 could also be a fluorescent polymer that emits at a different wavelength than polymer 90. By calculating the ratio of the two signals, it is possible to eliminate the effects of varying skin absorption and changes in sensor-detector distance. Instead of fluorescent dyes, the sensor could use semiconductor quantum dots, which are less susceptible to degradation.

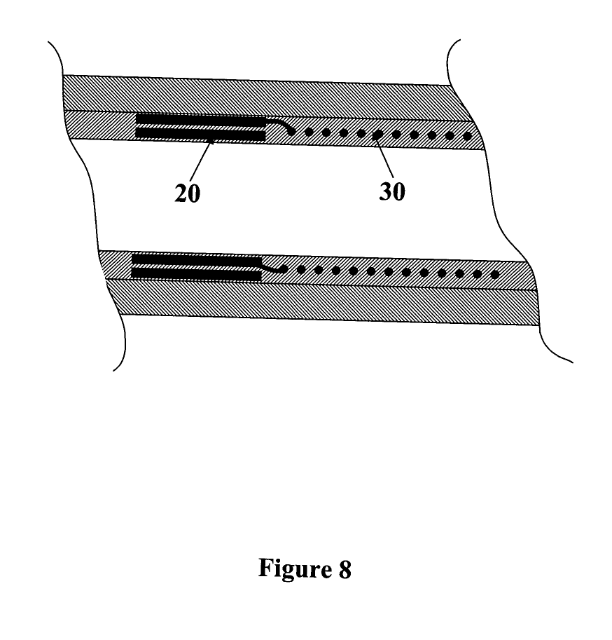

FIG. 8 shows a cross sectional view through the sensor area of another embodiment of the graft. The sensor elements are imbedded within two layers of biocompatible synthetic material, (e.g., expanded-polytetraflouroethylene (e-PTFE), polyurethane). Each layer of biocompatible material can have different mechanical properties and porosity to optimize the characteristic of the device as a graft. The sensor elements can be integrated between layers to simplify manufacturing. Although the inductive coil 30 is shown as coaxial to the graft it could also be positioned as 30 shown in FIG. 4.

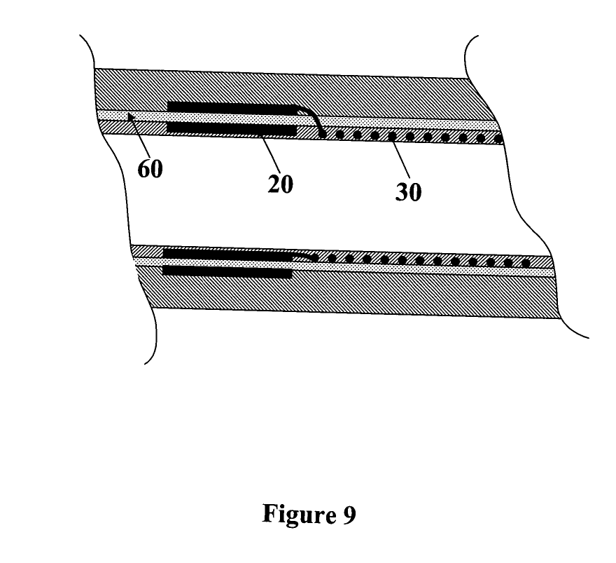

FIG. 9 shows a cross sectional view through the sensor area of another embodiment of the graft. The sensor elements are imbedded in a three layer synthetic graft. Each layer of biocompatible material (e.g., expanded-polytetraflouroethylene (e-PTFE), polyurethane) can have different mechanical properties and porosity. In this embodiment, the middle layer 60 acts as the dielectric between the plates of the cylindrical capacitor pressure sensor 20. In an alternative embodiment, the sensor elements are integrated between layers to simplify manufacturing. Although the inductive coil 30 is shown as coaxial to the graft, it could also be positioned as 30′ shown in FIG. 4.

The sensor elements described in the various embodiments can be integrated into the graft using injection and molding techniques commonly known in the art. For one embodiment the key fabrication steps for making the graft are: First a tube of ePTFE with wall thickness of approximately 0.5 mm and inside diameter of 6 mm is placed over a cylindrical stainless steel mandrel. This ePTFE has a large internodal distance (40-200 μm) to enhance cell endothelization along the inner surface and enable tissue ingrowth. The sensor element is then dilated to fit over the inner tube and placed near the end of the graft. The exact placement depends on the length of graft required for suturing. When released the sensor makes intimate contact with the inner surface. Finally, an outer tube of ePTFE is then tightly disposed over the inner tube and sensor. The outer tube ePTFE has a smaller internodal distance (15-35 μm) which results in higher mechanical strength. The complete assembly is then sintered at a temperature of about 350° C. for 15-30 minutes to bond the two tubes. The ePTFE, with a wide range of internodal distances, is available, e.g., from ZEUS (Orangeburg, S.C.).

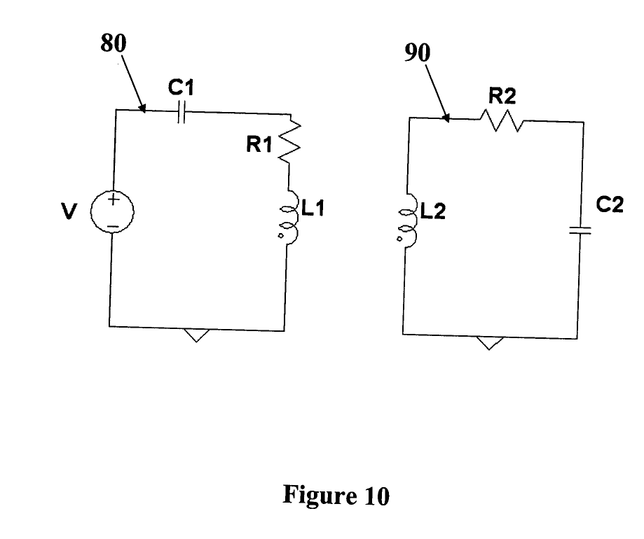

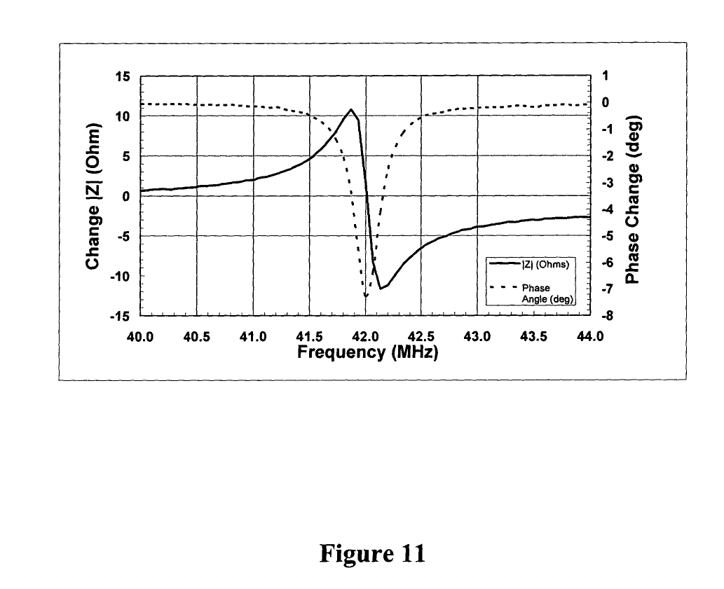

FIG. 10 shows an effective circuit model for an external remote detector 80 and passive sensor 90. Passive sensor 80 represents the embodiments of FIGS. 2-5, 8 and 9. The external remote detector 80 is modeled as a circuit with an effective inductance, L1, capacitance, C1, and resistance, R1. The passive sensor 90 is modeled as a circuit with an effective inductance, L2, capacitance, C2, and resistance, R2. Mutual inductance between the two circuits allows the external remote detector 80 to measure the resonance frequency of the passive sensor 90. FIG. 11 shows the change in complex impedance (amplitude and phase) measured by the external remote detector for a prototype passive sensor. The resonance frequency, which occurs at the minimum in phase angle, is clearly visible at 42 MHz.

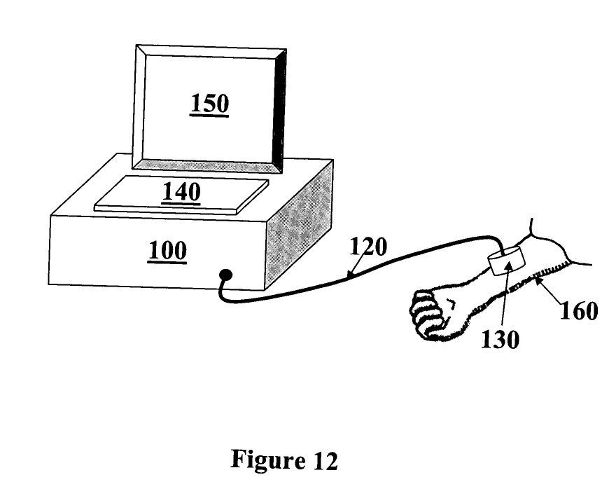

FIG. 12 shows the major components of the measurement system. A control electronics module 100 connects to the measurement probe 130 through a cable 120. In normal use, the control electronics module 100 collects sensor signals using probe 130 and processes the data for display on monitor 150. The probe 130 is placed over the arm 160 near the sensor elements. User interface 140 is used to control data acquisition, data display and analysis. In an alternative embodiment, the measurement probe and electronics are integrated into a compact handheld device that can be easily moved from patient to patient.

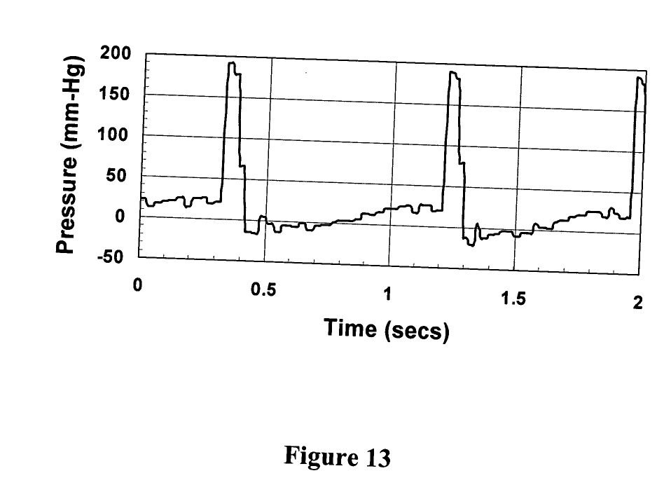

FIG. 13 shows the pressure pulse for multiple cycles as measured using the present invention. These results were obtained using a sensor configuration as described in FIG. 4. Pulsatile flow was produced using a Harvard Apparatus Model 1421 Pulsatile Blood Pump. The measured pressure pulse of FIG. 13 reproduced the intragraft pressure pulse as measured with a SenSym SCX05DN pressure sensor.

The above descriptions and illustrations are only by way of example and are not to be taken as limiting the invention in any manner. One skilled in the art can substitute known equivalents for the structures and means described. The full scope and definition of the invention, therefore, is set forth in the following claims.

We claim:

1. An apparatus, comprising:
a tubular structure of biocompatible material;
a first passive sensor element imbedded within said tubular structure, wherein said first passive sensor element produces a first signal that is a function of liquid pressure within said tubular structure; and
a second passive sensor element imbedded within said tubular structure at a distance from said first passive sensor element, wherein said second passive sensor element produces a second signal that is a function of liquid pressure within said tubular structure.

2. The apparatus of claim 1, further comprising means for collecting and analyzing said first signal and said second signal to determine the location or extent of an occlusion within or near said tubular structure.

3. The apparatus of claim 1, wherein said biocompatible material is selected from the group consisting of expanded polytetrafluorethylene (ePTFE) and polyurethane.

4. The apparatus of claim 1, wherein said tubular structure comprises a single layer of said biocompatible material.

5. The apparatus of claim 1, wherein said first passive sensor element and said second passive sensor element each comprise a capacitive element, C, and an inductive element, L, that forms an LC circuit, wherein said LC circuit comprises an impedance that becomes totally resistive at a characteristic resonance frequency, ω=1/\sqrt{LC}.

6. The apparatus of claim 5, wherein said first passive sensor element and said second passive sensor element each are designed such that said characteristic resonance frequency changes as said liquid pressure within said tubular structure changes.

7. The apparatus of claim 1, wherein said first passive sensor element comprises a first characteristic resonance frequency and said second passive sensor element comprises a second characteristic resonance frequency that is selected to be different from said first characteristic resonance frequency to easily separate the signals during measurement.

8. The apparatus of claim 2, wherein said means for collecting and analyzing said first signal and said second signal comprises an external pickup coil adapted for detecting the resonance frequency of said first passive sensor element and said second passive sensor element.

9. The apparatus of claim 1, wherein said first passive sensor element and said second passive sensor element are each adapted to have a response time of less than 100 msec.

10. The apparatus of claim 1, wherein said first passive sensor element and said second passive sensor element are each adapted to have a resonance frequency within a range of 1 MHz to 200 MHz.

11. The apparatus of claim 1, wherein said tubular structure comprises at least two layers of biocompatible material.

12. The apparatus of claim 11, wherein said first passive sensor element and said second passive sensor element are integrated between layers of said at least two layers.

13. The apparatus of claim 1, wherein said first passive sensor element and said second passive sensor element are imbedded within said tubular structure.

14. The apparatus of claim 5, wherein said capacitive element comprises a cylindrical capacitor having plates with a spacing that is a function of said liquid pressure within said tubular structure.

15. The apparatus of claim 14, wherein said cylindrical capacitor comprises a split cylindrical capacitor.

16. The apparatus of claim 14, wherein said cylindrical capacitor comprises a dielectric fluid trapped within said cylindrical capacitor to produce a trapped volume that is adapted to increase the pressure sensitivity of said first passive sensor element and said second passive sensor element.

17. The apparatus of claim 5, wherein said inductive element comprises a coil that wraps around said tubular structure.

18. The apparatus of claim 5, wherein said inductive element comprises a coil placed on the top surface of said tubular structure, wherein said coil comprises an axis oriented to be perpendicular to axis of said tubular structure.

19. The apparatus of claim 1, wherein at least one of said first passive sensor element and said second passive sensor element comprises an optical pressure sensor element having an optical property that changes as a function of said liquid pressure within said tubular structure.

20. The apparatus of claim 19, wherein said optical pressure sensor element comprises a Fabry-Perot filter element having a mirror spacing that changes as a function of said liquid pressure within said tubular structure.

21. The apparatus of claim 19, further comprising a broadband light source adapted to illuminate said optical pressure sensor element and produce a return signal.

22. The apparatus of claim 21, further comprising a spectrometer adapted to measure said return signal to detect the operating wavelength of said filter.

23. The apparatus of claim 22, wherein said filter is designed such that said return signal is either a maximum or minimum at an operating wavelength of said filter.

24. The apparatus of claim 23, wherein said filter is designed to have an operating wavelength within a range from 600 nm and 1200 nm.

25. The apparatus of claim 21, further comprising a fluorescent material positioned and adapted to produce a fluorescent signal upon interaction with light from said broadband light source, wherein said fluorescent signal will interact with said filter.

26. A method, comprising:
providing a tubular structure of biocompatible material;
imbedding a first passive sensor element within said tubular structure, wherein said first passive sensor element is adapted to produce a first signal that is a function of liquid pressure within said tubular structure; and
imbedding a second passive sensor element within said tubular structure at a distance from said first passive sensor element, wherein said second passive sensor element is adapted to produce a second signal that is a function of liquid pressure within said tubular structure.

27. The method of claim 26, further comprising providing means for collecting and analyzing said first signal and said second signal to determine the location or extent of an occlusion within or near said tubular structure.

28. The method of claim 26, wherein said first passive sensor element and said second passive sensor element each comprise a capacitive element, C, and an inductive element, L, that forms an LC circuit, wherein said LC circuit comprises an impedance that becomes totally resistive at a characteristic resonance frequency, ω=1/\sqrt{LC}.

29. The method of claim 28, wherein said first passive sensor element and said second passive sensor element each are designed such that said characteristic resonance frequency changes as the blood pressure within said tubular structure changes.

30. An method for determining the location or extent of an occlusion within or near a tubular structure, wherein said a tubular structure comprises biocompatible material and includes a first passive sensor element imbedded within said tubular structure, wherein said first passive sensor element is adapted to produce a first signal that is a function of liquid pressure within said tubular structure, wherein said tubular structure further comprises a second passive sensor element imbedded within said tubular structure at a distance from said first passive sensor element, wherein said second passive sensor element is adapted to produce a second signal that is a function of liquid pressure within said tubular structure, the method comprising collecting and analyzing said first signal and said second signal to determine the location or extent of an occlusion within or near said tubular structure.

31. The method of claim 30, wherein the step of collecting and analyzing said first signal and said second signal is performed with an external pickup coil adapted for detecting the resonance frequency of said first passive sensor element and said second passive sensor element.
